(19) United States
(12) Patent Application Publication
(10) Pub. No.: US 2026/0335252 A1
(43) Pub. Date: Oct. 29, 2026

(54) CELLULOSE NANOFIBRIL-BASED HYDROGELS CONTAINING WOUND HEALING ADJUVANTS

(71) Applicants: The United States of America, as represented by the Secretary of Agriculture, Washington, DC (US); Obatala Sciences, New Orleans, LA (US)

(72) Inventors: Nicolette T. Prevost, New Orleans, LA (US); Michael W. Easson, New Orleans, LA (US); Jacobs Harris Jordan, New Orleans, LA (US); Judson V. Edwards, New Orleans, LA (US); Cecilia G. Sanchez, New Orleans, LA (US); Trivia Frazier, New Orleans, LA (US)

(21) Appl. No.: 19/193,179

(22) Filed: Apr. 29, 2025

(51) Int. Cl. A61K 9/70; A61K 9/06; A61K 38/18; A61K 38/19; A61K 38/39; A61K 47/38; A61P 17/02

(52) U.S. Cl. CPC A61K 9/70; A61K 9/06; A61K 38/18; A61K 38/19; A61K 38/39; A61K 47/38; A61P 17/02

(57) ABSTRACT

Compositions and methods for treating wound healing are described using compositions comprising cellulose nanofibrils (CNFs) and hydrogels. Exemplified hydrogel-CNF compositions include blood-derived extracellular matrix-hydrogels comprising blood-derived growth factors, cytokines and ECM proteins wherein the treatment supports proliferation, differentiation, and migration capacity in wound healing and tissue regeneration.

### BACKGROUND OF THE INVENTION

Numerous factors can pose challenges to the wound healing process. These factors include poor circulation, unresolved inflammation, neuropathy, infection, lack of angiogenesis, lack of cell proliferation, etc. Acute and chronic skin wounds due to burns, pressure injuries, and trauma represent a substantial challenge to healthcare delivery with particular impacts on geriatric, paraplegic, quadriplegic among other demographics worldwide. Current standard of care relies extensively on preventive measures to mitigate pressure injury, surgical debridement, skin flap procedures, and negative pressure wound vacuum measures.

Employing efficient wound care treatment is important to ensure a wound has the best chance of healing. There is an ongoing need to develop efficacious wound healing compositions and methods which are also cost-effective, sustainable, and easily adaptable to widespread use.

### SUMMARY OF THE INVENTION

Described herein in various embodiments are compositions and methods for wound healing; exemplified compositions comprising, cellulose nanofibrils (CNFs) and a hydrogel of blood-derived extracellular matrix (ECM) proteins are described.

In one embodiment, the composition comprises cellulose nanofibrils (CNFs) and at least one mammalian blood-derived extracellular matrix protein. In another embodiment, the composition comprises cellulose nanofibrils (CNFs) and a human blood-derived extracellular matrix (ECM) protein.

In one embodiment, the composition comprises cellulose nanofibrils (CNFs) and at least one of growth factors, cytokines and ECM proteins. In another embodiment, the growth factors in the composition are human blood-derived growth factors.

In one embodiment, the growth factors, cytokines and ECM proteins include collagen and fibronectin.

In one embodiment, the proteins comprising the compositions and methods of treatment described herein are selected from one or more of human serum albumin, fibrillin-1, a2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1.

Described herein in various embodiments are methods of treating a wound comprising treatment with an effective amount of a composition comprising cellulose nanofibrils (CNFs) and blood-derived extracellular matrix-hydrogel.

In one embodiment, a method of treating a wound comprising treatment with an effective amount of a composition comprising cellulose nanofibrils (CNFs) and a extracellular matrix-hydrogel comprising at least one of growth factors, cytokines and/or ECM proteins. In another embodiment, the growth factors in the composition are human blood-derived growth factors.

In one embodiment, the growth factors, cytokines and ECM proteins include collagen and fibronectin.

In one embodiment, a method of treating a wound is described comprising treatment with an effective amount of a composition comprising cellulose nanofibrils (CNFs) and blood-derived extracellular matrix-hydrogel comprising blood-derived growth factors, cytokines and ECM proteins wherein the treatment supports proliferation, differentiation, and migration capacity in the wound healing process. In another embodiment, the wound healing is cutaneous wound healing. In another embodiment, the wound healing is tissue regeneration. In one embodiment, the wound healing is subcutaneous tissue regeneration. In one embodiment, the wound healing is subcutaneous and epithelial regeneration. In another embodiment, the wound healing is reestablishment of subcutaneous and epithelial regeneration.

### BRIEF DESCRIPTION OF THE DRAWINGS

FIG. 1 shows visual presentation of hydrogel #4 formulation (see table 1) for TOCNF, TOCNF (3:1) dilution, CNF and CNF (3:1) dilution after 30 minutes at 37 °C.

FIG. 2 shows an exemplified (ObaGel®-ECM) proteome profile demonstrating an extracellular matrix protein dominant composition.

FIG. 3 shows graphical representation of the rheological data.

FIG. 4 is SEM imaging and analysis of CNF and TOCNF hydrogel mixtures exhibiting similar microstructures.

FIG. 5 shows Biocompatibility, dermal fibroblast cells (DFCs) cultured on the surface or encapsulated in the CNF and TOCNF hydrogels

FIG. 6 shows adipose-derived stromal/stem cells (ASCs) exhibited similar lipid deposition on TOCNF and CNF hydrogels.

FIG. 7. shows a wound healing assay. ASC migratory behavior.

### DETAILED DESCRIPTION

Described herein, compositions and methods for treating wound healing using compositions comprising cellulose nanofibrils (CNFs) and hydrogels are described.

Exemplified hydrogel-CNF compositions and methods described include blood-derived extracellular matrix-hydrogels comprising blood-derived growth factors, cytokines and ECM proteins wherein the treatment supports proliferation, differentiation, and migration capacity in wound healing and tissue regeneration.

In one embodiment, compositions comprising cellulose nanofibrils (CNFs) and a hydrogel of blood-derived extracellular matrix are described.

In one embodiment, the composition comprises cellulose nanofibril (CNFs) and a mammalian blood-derived extracellular matrix. In another embodiment, the composition comprises cellulose nanofibril (CNFs) and at least one proteins derived from human blood-derived extracellular matrix.

In one embodiment, the composition comprises cellulose nanofibril (CNFs) and at least one of growth factors, cytokines and/or ECM proteins. In another embodiment, the growth factors are blood derived growth factors.

In one embodiment, the composition comprises a homogenous hydrogel comprising cellulose nanofibril and extracellular matrix.

In one embodiment, the composition comprises a homogenous hydrogel comprising 0.01% to 10% w/v cellulose nanofibril in extracellular matrix. In another embodiment, the composition comprises a homogenous hydrogel comprising 0.5% to 1% w/v cellulose nanofibril in extracellular matrix. In another embodiment, the composition comprises a homogenous hydrogel comprising 0.3% to 0.77% w/v cellulose nanofibril in extracellular matrix. As used herein, concentrations above are for weight percent or percent weight per volume % (w/v) units are grams/100 mL and 1%(w/v)=1 gram/100 mL In another embodiment, the growth factors, cytokines and ECM proteins include collagen and fibronectin.

In one embodiment, the proteins comprising the compositions and methods of treatment described herein are selected from one or more of human serum albumin, fibrillin-1, a2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1.

In one embodiment, the cellulose nanofibrils used have dimensions of about 2-200 nm in cross sections and dimensions of about 0.01-200 μm in length. In another embodiment, the cellulose nanofibrils used have dimensions of about 4-100 nm in cross section and dimension of about 0.1-20 μm in length.

In one embodiment, the cellulose nanofibril used are prepared from a cellulose obtained from a plant which is optionally chemically modified to form a cellulose nanofibril exemplified by TEMPO-oxidized cellulose nanofibrils (TOCNF).

In one embodiment, the cellulose nanofibril used is prepared from wood or cotton.

In one embodiment, the nanofibrils used are TEMPO-oxidized cellulose nanofibrils (TOCNF). In another embodiment, the nanofibrils used are cellulose nanofibrils (CNF).

Described herein in various embodiments are methods of treating a wound comprising treatment with an effective amount of a composition comprising cellulose nanofibrils (CNFs) and blood-derived extracellular matrix-hydrogel.

In one embodiment, a method of treating a wound comprising treatment with an effective amount of a composition comprising cellulose nanofibrils (CNFs) and blood-derived extracellular matrix-hydrogel comprising blood-derived growth factors, cytokines and ECM proteins. In another embodiment, the method of treatment optionally includes stem cells.

In one embodiment, the growth factors, cytokines and ECM proteins include collagen and fibronectin.

In one embodiment, a method of treating a wound is described comprising treatment with an effective amount of a composition comprising cellulose nanofibrils (CNFs) and blood-derived extracellular matrix-hydrogel comprising blood-derived growth factors, cytokines and ECM proteins wherein the treatment supports proliferation, differentiation, and migration capacity in the wound healing process. In another embodiment, the wound healing is cutaneous wound healing. In another embodiment, the wound healing is tissue regeneration. In another embodiment, the wound healing is subcutaneous tissue regeneration. In another embodiment, the wound healing is subcutaneous and epithelial regeneration. In another embodiment, the wound healing is reestablishment of subcutaneous and epithelial regeneration.

In one embodiment, a composition comprising cellulose nanofibrils and at least one hydrogel wherein the hydrogel comprises an extracellular matrix (ECM) is described. In another embodiment of the composition, the cellulose nanofibril (CNF) is a modified TEMPO oxidized cellulose nanofibril (TOCNF). In another embodiment of the composition, the hydrogel is amended with growth factors, cytokines and extracellular matrix proteins. In another embodiment, the hydrogel is amended with growth factors, cytokines and one or more extracellular matrix protein selected from the group consisting of a collagen, elastin fibronectin, laminin, glycoproteins, tenascin, fibrillin, proteoglycans and glycosaminoglycans.

In another embodiment of the composition, the hydrogel comprises of a blood-derived growth factor. In another embodiment of the composition, the hydrogel comprises of a cytokine. In another embodiment of the composition, the extracellular matrix proteins include collagen and fibronectin. In another embodiment of the composition, the hydrogel comprises extracellular matrix proteins in a range of 50 to 80% in respect to the amount of nanofibrils added to the resulting mixtures In another embodiment of the composition, the hydrogel comprises a proteome which supports proliferation, differentiation, and migration capacity in wound healing and tissue regeneration. In another embodiment of the composition, the hydrogel includes at least one protein selected from human serum albumin, fibrillin-1, a2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1 or mixtures thereof. In another embodiment of the composition, the hydrogel comprises a proteome profile of a extracellular matrix protein composition including growth factors, cytokines and extracellular matrix proteins.

In one embodiment, a method of treating a wound with an effective amount of a composition comprising: cellulose nanofibrils and at least one hydrogel wherein the hydrogel comprises an extracellular matrix. In another embodiment of the method, the cellulose nanofibril (CNF) is a modified TEMPO oxidized cellulose nanofibril (TOCNF). In another embodiment of the method, the hydrogel is amended with extracellular matrix proteins. In another embodiment of the method, the hydrogel comprises of blood-derived growth factors. In another embodiment of the method, the hydrogel comprises of cytokines. In another embodiment of the method, the hydrogel comprises of blood-derived growth factors, cytokines and extracellular matrix proteins in the range for effective wound healing in a subject. In another embodiment of the method, the hydrogel includes at least one protein selected from human serum albumin, fibrillin-1, α2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1. In another embodiment of the method, the hydrogel comprises a proteome which supports proliferation, differentiation, and migration capacity in wound healing and tissue regeneration. In another embodiment of the method, the wound is a cutaneous wound. In another embodiment of the method, the wound is a burn wound. In another embodiment of the method, the wound is a chronic wound. In another embodiment of the method, the wound is a wound in a diabetic patient. In another embodiment of the method, the wound treated is decubitus ulcer. In another embodiment of the method, the wound is a venous ulcer.

In one embodiment a method of synthesizing a wound healing composition is described comprising the steps of: preparing a solution of cellulose nanofibrils in water, adjusting the solutions to a required pH, and, adding a extracellular matrix (ECM) hydrogel to the nanocellulose fibril solution. In another embodiment of the method, the cellulose nanofibril (CNF) is a modified CNF, TEMPO oxidized cellulose nanofibrils (TOCNF). In another embodiment of the method, the ECM hydrogel includes at least one protein selected from human serum albumin, collagen, ibrillin-1, α2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1. In another embodiment of the method, the hydrogel is amended with extracellular matrix proteins. In another embodiment of the method, the hydrogel comprises of cytokines. In another embodiment of the method, the hydrogel comprises of blood-derived growth factors, cytokines and ECM proteins.

Abbreviations used herein include, CNF—Cellulose nanofibrils; ECM—extracellular matrix; ASC—adipose—derived stromal/stem cells; DFC—dermal fibroblast cells; CNC—cellulose nanocrystals; DAT—decellularized adipose tissue; TOCNF—TEMPO-2,2,6,6-tetramethylpiperidine-1-oxyl-oxidized cellulose nanofibrils; PBS—phosphate buffered saline; CO₂—carbon dioxide; RIPA—radioimmunoprecipitation assay; BSA—Bovine Serum Albumin; SEM—Scanning Electron Microscopy; AFM—Atomic Force Microscopy. ALB—human serum albumin; FBN—1—fibrillin-1, A2M—α2-macroglobulin; APOA1—apolipoprotein AI; SERPINA1—α1—antitrypsin; APOB—apolipoprotein B: COL1A1—alpha-1 type I collagen; COL1A2—collagen alpha-2(I) chain; CP—ceruloplasmin (or caeruloplasmin); CPH—carboxypeptidase H; FLNA—filamin A, alpha; APOA4—apolipoprotein A-IV; ACTB—actin beta; COL3A1—Type III collagen; COL6A3=Type VI collagen alpha-3(VI) chain; FBN2—fibrillin-2; FN1—fibronectin; KRT1—keratin 1.

While the following terms in relation to the presently disclosed compounds are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The term cellulose nanofibrils (CNF) or nanocellulose fibril describes cellulosic fiber materials that have at least one of their dimensions in the nanoscale. Cellulose nanofibrils usually have a high aspect ratio (length to width ratio). CNF are flexible and are often only nm-thick in diameter yet can be microns in length, providing a significant surface area to volume ratio. TEMPO-oxidized CNF (TOCNF) also known as 2,2,6,6-tetramethylpiyperidine-1-oxyl (TEMPO)-oxidized cellulose nanofibrils refers to cellulose nanofibrils that have been chemically modified by oxidation using an oxidizing reagent compound called 2,2,6,6-tetramethylpiperidine-1-oxyl (TEMPO), which introduces carboxyl groups onto the cellulose surface.

Hydrogel refers to a biphasic material, a mixture of porous and permeable solids and at least 10% of water or other interstitial fluid. The solid phase is a water insoluble three-dimensional network of polymers wherein the network of crosslinks which bond the polymers of a hydrogel fall under two general categories: physical hydrogels and chemical hydrogels with chemical hydrogels having covalent cross-linking bonds and physical hydrogels have non-covalent bonds. A hydrogel may absorb a large amount of water or biological fluid.

Extracellular matrix refers to a network consisting of extracellular macromolecules and minerals, such as collagen, enzymes, glycoproteins and hydroxyapatite that provide structural and biochemical support to surrounding cells. Some exemplified ECM proteins used herein and their functions are: Collagen which is the most abundant protein in the ECM, providing tensile strength and structural support. 28 types of collagen have been identified and classified into 5 types. Elastin which provides elasticity and allows tissues to stretch and recoil. Fibronectin, which is an adhesive glycoprotein that helps cells attach to the ECM. Laminin, which is another adhesive glycoprotein, particularly important in basement membranes. Glycoproteins which are large protein-carbohydrate molecules that play various roles in cell adhesion and signaling. Tenascin, which is an adhesive protein involved in tissue organization and cell migration. Fibrillin, which forms microfibrils, providing support to elastic tissues. Proteoglycans which are glycoproteins with glycosaminoglycan chains that provide hydrated gel-like structure and resist compressive forces.

Glycosaminoglycans (GAGs), which are negatively charged polysaccharides that interact with ECM proteins and growth factors.

In another embodiment, the growth factors are blood derived growth factors.

A growth factor refers to a naturally occurring substance exemplified by a protein or a steroid hormone that regulates a variety of cellular processes including stimulating cell proliferation, wound healing, and occasionally cellular differentiation. It may have a protein, steroid hormone or other molecular structure.

A cytokine refers to type of protein that is made by certain immune and non-immune cells and has an effect on the immune system. Some cytokines stimulate the immune system and others slow it down. They can also be made in the laboratory and used to help the body fight cancer, infections, and other diseases.

The term proteome refers to the assortment of proteins. One example is the assortment of proteins produced at a specific time in a particular cell or tissue type. A proteome may be prepared by a cell or tissue, etc. or other means available in biotechnology.

Amending refers to changing or modifying by adding or removing a constituent from a composition. For example, one or more proteins may be added to or removed from a composition thus amending it.

Wound healing refers to a complex and dynamic process of replacing devitalized and missing cellular structures and tissue layers that can be described by a process which can be divided into 3 (inflammatory, proliferation, and remodeling) or 4 (the hemostasis phase, the inflammatory phase, the proliferation phase, and the remodeling phase) phases.

A cutaneous wound refers to a disruption of normal anatomic structure and function of the skin that occurs owing to an injury of the skin.

A chronic wound refers to injured tissue that won't heal within normal time frames.

A decubitus ulcer refers to injury to skin and underlying tissue resulting from prolonged pressure on the skin.

A venous ulcer refers to a wound that takes longer than usual to heal due to vein and blood flow issues.

The terms ObaGel®, ObaGel®-ECM, and decellularized adipose tissue(DAT) (AdipoGel™) are names of human-derived hydrogels developed for applications including wound healing and regenerative medicine. ObaGel® is the first commercially available human blood-derived 3D culture scaffold capable of supporting and promoting robust adipogenesis and stimulating the formation of vascular-like structures. Likewise, ObaGel®-ECM and DAT hydrogels are reported to support stem cell proliferation, multi-lineage differentiation, and remodeling that mimic in vivo adipose tissue microstructure.

The compositions described herein are useful in the treatment, amelioration and/or prevention of the pathological condition as described herein in a animal or human in need thereof. The term “treatment” refers to both therapeutic treatment and prophylactic or preventative measures. Treatment includes the application or administration of a composition to the body, an isolated tissue, or cell from a patient which has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “effective amount” of a composition, compound or property as provided herein is meant by such amount as is capable of performing the function of the compound or property or treatment for which an effective amount is expressed. As is pointed out herein, the exact amount required will vary from process to process, depending on recognized variables such as the compounds employed, and various internal and external conditions observed as would be interpreted by one of ordinary skill in the art. An appropriate effective amount may be determined, however, by one of ordinary skill in the art using only routine experimentation.

The amounts, percentages, and ranges disclosed herein are not meant to be limiting, and increments between the recited amounts, percentages, and ranges are specifically envisioned as part of the invention.

The term “optional” or “optionally” means that the subsequently described event or circumstance may or may not occur, and that the description includes instances in which said event or circumstance occurs and instances where it does not. For example, the phrase “optionally in a solvent” means that the composition may or may not need a solvent and that this description includes compositions that contain and do not contain a solvent.

In specific embodiments, narrower ranges of the amounts of each component may be desired for a particular use or characteristic in the final product.

The term “substantially pure” refers to a formulation that is at least about 90% (e.g., at least 90%) in purity weight/weight of a total composition. In a more preferred embodiment, the purity is at least about 95% (e.g., at least about 95%) weight-to-weight, or at least about 98% (e.g., at least about 98%) purity.

As used herein, the term “about” is defined as plus or minus ten percent of a recited value.

For example, about 1.0 g means 0.9 g to 1.1 g.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

It will be obvious to those skilled in the art that embodiments described herein are provided by way of example only. Numerous variations, changes, and substitutions will occur to those skilled in the art without departing from the embodiments of the claims. Various alternatives to the embodiments of the claims described herein may be employed in practicing the use of compositions and methods of treatment described herein. It is intended that the included claims define the scope of the various compositions and methods of treatment described herein and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

The term “consisting essentially of” excludes additional method (or process) steps or composition components that substantially interfere with the intended activity of the method (or process) or composition. This term may be substituted for inclusive terms such as “comprising” or “including” to define any of the disclosed embodiments or combinations/sub-combinations thereof. Furthermore, the exclusive term “consist of” is also understood to be substitutable for these inclusive terms.

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention.

As used herein, the word “exemplified or exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation. Unless defined otherwise, all technical and scientific terms used herein have the same meaning as commonly understood by one of ordinary skill in the art to which the invention belongs.

Although any methods and materials similar or equivalent to those described herein can be used in the practice or testing of various embodiments of the inventions described, exemplified methods and materials are now described.

### EXAMPLES

Having now generally described the compositions, methods of preparation and other embodiments described herein, the same will be better understood by reference to certain specific examples, which are included herein only to further illustrate the embodiments and are not intended to limit the scope of the same as defined by the claims. Mention of trade names or commercial products herein is solely to provide specific information and does not imply recommendation or endorsement by the U.S. Department of Agriculture.

### Materials

Obatala Sciences ObaGel®-ECM A (cat #OS-301); Obatala Sciences ObaGel®-ECM B (cat #OS-314) are proprietary human blood-derived hydrogel extracellular matrix; 2,2,6,6-tetramethylpiperidine-1-oxyl (TEMPO)-oxidized cellulose nanofibrils (TOCNF) and cellulose nanofibrils (CNF) were obtained from The University of Maine Process Development Center as freeze-dried powder and was stored at room temperature until use. Cotton CNF (SRRC, USDA) were isolated from cotton gin mote cellulose by alkaline processing, acidified chlorite bleaching, and high-pressure homogenization as described previously and was used as an one weight percent suspension. General chemical reagents were used as received.

### Fabrication of Hydrogel Blends

Stock solutions, 1% (w/v) or 10 mg/mL, of cellulose nanofibrils(CNF) and TEMPO oxidized cellulose nanofibrils(TOCNF) were made in water (deionized, cell culture grade). These stock solutions were neutralized and equilibrated to a final pH of 7.5 with 10× phosphate buffered saline solution (PBS, 4 mL). Four ratio blends were made of each nanocellulose fibril type with ObaGel®-ECM A and B, 5:1:4, 3:1:6; 24:1:25 and 1:2:2, in addition to the nanocellulose fibril solution alone as the control (Table 1) for gelation. ObaGel was added directly from the final product bottle (100%).

An aliquot of each hybrid hydrogel formulation and controls, 500 μL, were transferred to a 24-well microplate and incubated for 30 min. at 37 °C. Gelation of each hydrogel combination was visually assessed and documented as yes (Y) or no (N) (Table 1).

TABLE 1
Composition of the hydrogel/cellulose nanofibril based scaffolds^{a} prepared
and its gelation results after 30-minute incubation period at 37 °C.

| | | Hydrogel/CNF based combination scaffold | | | |
|---|---|---|---|---|---|
| | | composition ratio % | | | Gelation in |
| | % (w/v) | CNF or | ObaGel ® - | ObaGel ® - | 30 min |
| Embodiment | nanofibrils | TOCNF % | ECM A % | ECM B % | (Y/N) |
| 1 | 1 | 100 | 0 | 0 | N |
| 2 | 0.5 | 50 | 10 | 40 | Y |
| 3 | 0.30 | 30 | 10 | 60 | Y |
| 4 | 0.48 | 48 | 2 | 50 | Y |
| 5 | 0.2 | 20 | 40 | 40 | Y |
| 6 (diluted | 0.15 | 15 | 30 | 30 | Y |
| 5)^{b} | | | | | |
| 7 Oba Gel- | 0 | 0 | c | c | |
| ECM | | | | | |

^{a}Combination hydrogels were prepared with single nanocellulose fibril solution (cellulose nanofibril (CNF) or TEMPO- oxidized cellulose nanofibril (TOCNF)) in combination with ObaGel ®-ECM A and ObaGel ®-ECM B, ObaGel ®-ECM B alone, or in combination with reduced ObaGel ®-ECM A.
^{b}The diluted combination hydrogel #5 (ObaGel-ECM with either CNF or TOCNF) with 1X phosphate buffered solution (PBS) in a 3:1 ratio (75% of #5 and 25% of PBS).
c This embodiment is ObaGel-ECM only (50/50 A and B or 100% of A or 100% of B). Diluted #5 was determined the best formulation blend for both TOCNF and CNF based on characterization and biocompatibility data.

### Rheological Analysis

All rheological measurements were performed using a Discovery HR-2 rheometer (Waters| TA Instruments, New Castle, DE, USA). The stiffness, viscosity, and sol-gel transition were analyzed using a 25 mm parallel plate, final gap width of 500 μm, and a volume of 350 μL of each hydrogel formulation sample per run. Viscosity was measured from 1 to 100 s⁻¹ at 4 °C. Then, stiffness was measured at 0.1 to 100 Hz at 1% strain, (n=3). The temperature was initially ramped from 4 to 37 °C, waiting for 30 minutes, to stimulate crosslinking prior to collecting data points. G′ and G″ were evaluated at 1% strain from 0.1 to 100 Hz, 10 pts/decade. Sol-gel transition was determined for hydrogel formulation blends considered optimal based on hydrogel characterization and biocompatibility data. Samples was measured for 1800 s post-soak at 37 °C at 1 Hz and 1% strain. The temperature was increased from 4 to 37 °C at 1 °C·min⁻¹.

### Degradation Study

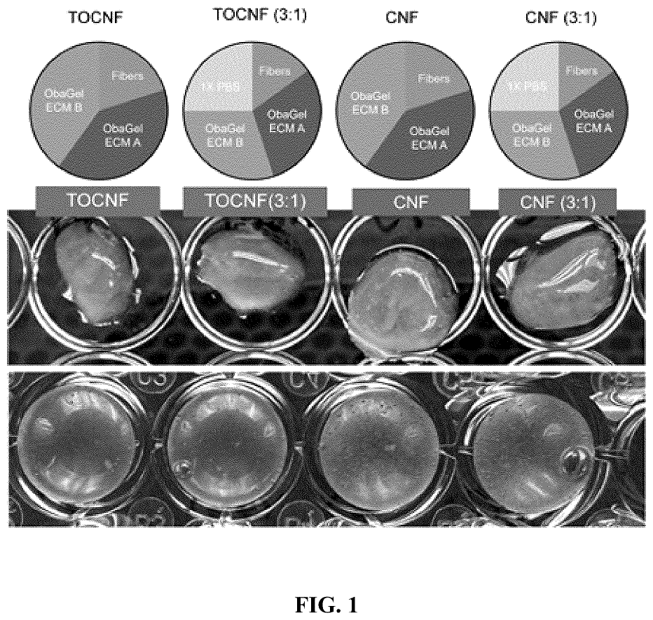

Each hydrogel formulation (250 μL) in duplicate was transferred to the inner middle wells of a 48-well microplate (FIG. 1). Sterile, deionized water was added to the perimeter wells of the plate. The plate was then incubated at 37 °C in a carbon dioxide (CO₂) incubator (ThermoForma, Model #3110) set at 5% CO₂ for 15 minutes to initiate gelation. Once the constructs gelled, 250 μL of 1×PBS was added to each well to the surface of the constructs. On days 0, 1, 2, and 7, 50 μL of the liquid interface was removed and replaced with fresh 1×PBS. The liquid substrate removed from the constructs was transferred to microtubes and placed in the freezer (−20 °C) for later analysis. The substrate aliquots collected on the selected four days were evaluated for protein leaching. BCA protein assay kit (Thermofisher, cat #23225) was used to quantify the amount of protein released into the PBS solution.

### Hydrogel Protein Content

Pierce BCA protein assay kit (Thermofisher, cat #23225) was used to quantify the protein content present in each hydrogel formulation. Select hydrogel formulations were aliquoted into microtubes and diluted 1:10 in radioimmunoprecipitation assay (RIPA) buffer (Sigma Aldrich, cat #R0278). Bovine Serum Albumin (BSA) standards and all reagents were prepared according to the manufacturer's protocol. Standards and samples were transferred to a labelled 96-well plate in triplicate. After adding 200 μL of the working reagent to each well, the plate was incubated at 37 °C in 5% CO₂ for 30 minutes. After cooling the plate to room temperature, the absorbance was measured at 560 nm using the Biotek Synergy H1 microplate reader (Agilent Santa Clara, CA, USA).

### Scanning Electron Microscopy (SEM)

Evaluation of hydrogel fiber diameter and porosity was done with SEM. All hydrogel formulations (150 μL) were transferred to a 48-well plate and incubated at 37 °C, 5% CO2 overnight. Samples were transported at room temperature to Tulane University Coordinated Instrument Facility (CIF) for analysis using the Hitachi S-4800 SEM (Hitachi High-Technologies Corporation, Japan) equipped with a Gatan Alto 2500 cryogenic SEM system (AMETEK, Berwyn, PA, USA), procedure described previously. Images were analyzed using the Image J Diameter J plugin to segment images and quantify fiber metrics-fiber diameter, intersection density and percentage porosity.

### Stem Cell Culture and Proliferation in 3D

Cryopreserved human adipose-derived stromal/stem cells (ASC) (Obatala Sciences, cat #OS-101) were thawed and counted according to Obatala Sciences' standard operating procedure. ASCs were resuspended in 1 mL of each hydrogel combination and seeded in 100 μL constructs at 100,000 cells/mL in a 96-well plate. At days one, two, and seven, 100 μL of a 1× alamarBlue™ Cell Viability Reagent solution (Invitrogen, cat #DAL1025) was added to the designated wells and incubated at 37 °C, 5% CO₂ for 4 hours. After incubation, 70 μL of the reduced samples were transferred to a clear-bottom, black, 96-well plate. Absorbance and fluorescence were measured at 570/600 nm and 560/590 (ex/em), respectively, using the Biotek Synergy H1 microplate reader.

### Dermal Fibroblast Proliferation on 2D Coating

Hydrogel mixtures of TOCNF #4 and CNF #4 were prepared at a 3:1 dilution (750 μL of the hydrogel mixture and 250 μL of 1×PBS). Twenty-four-well plates were coated with 250 μL of each hydrogel mixture and incubated at 37 °C, 5% CO₂ for 15 minutes to facilitate gelation. Thus ECM, TOCNF #4, CNF #4, and the 3:1 dilution of formulation #4 were coated on the plates. Human dermal fibroblast cells (DFC) (Obatala Sciences, cat #OS-106) were resuspended at 100,000 cells/ml in StromaQual™ (Obatala Sciences, cat #OS-001) and seeded at 10,000 cells/well on top of the hydrogels. StromaQual™ (150 μL) was added to each well after seeding cells and plate was returned to 37 °C, 5% CO2 incubator overnight. At days one, two, and seven, 500 μL of a 1× alamarBlue™ Cell Viability Reagent solution was added to the designated wells and incubated at 37 °C, 5% CO₂ for 4 hours. After incubation, 100 μL of the reduced samples were transferred to a clear-bottom, black, 96-well plate. Absorbance and fluorescence were measured at 570/600 nm and 560/590 (ex/em), respectively, using the Biotek Synergy H1 microplate reader. Remainder culture medium was removed from the wells and the DFCs were fixed with 10% neutral buffered formalin, washed with 1×PBS, stained with Hoechst 33342 (Thermofisher, cat #R37605), and imaged with Motic AE2000 Phase Contrast Microscope (Motic, Schertz, TX, USA).

### Dermal Fibroblast Proliferation in 3D

Cryopreserved human DFCs (Obatala Sciences, cat #OS-106) were thawed and counted according to Obatala Sciences' standard operating procedure. DFCs were resuspended at 100,000 cells/mL in StromaQual™. Volumes containing 200,000 cells were transferred to individual microcentrifuge tubes with 1 mL of each hydrogel mixture. The cell-hydrogel mixtures were transferred to a 96-well plate with a final concentration of 10,000 cells/well. At days one, two, three, and seven, 100 μL of a 1× alamarBlue™ Cell Viability Reagent solution was added to the designated wells and incubated at 37 °C, 5% CO₂ for 4 hours. After incubation, 70 μL of the reduced samples were transferred to a clear-bottom, black, 96-well plate. Absorbance and fluorescence were measured at 570/600 nm and 560/590 (ex/em), respectively, using the Biotek Synergy H1 microplate reader.

### Adipose-Derived Stromal/Stem Cells Biocompatibility Assay

Human adipose-derived stromal/stem cells (ASC)s (Obatala Sciences, cat #OS-101) were seeded at 89,000 cells/well of a PCL NanoECM® 24-well plate (Neuromics, cat #2401) coated with hydrogel mixtures described in Table 1. ASCs were maintained in StromaQual™ (Obatala Sciences, cat #OS-001) for 11 days followed by adipogenic differentiation with AdipoQual™ (Obatala Sciences, OS-002) for 31 days.

### Adipose-Derived Stromal/Stem Cells Wound Healing Assay

Human ASCs were seeded at 100,000 cells/well with or without inserts provided in the Wound Healing Assay kit (abeam, cat #ab242285) on 12-well plates coated with the following hydrogel mixtures: ObaGel®-ECM B, CNF #4, and TOCNF #4. Cultures were maintained at 37 °C, 5% CO₂ until the cells attained confluency. After confluency was attained, cells were fixed with 4% formalin and the cytoskeleton and nuclei were stained with Alexa Fluor Phalloidin 594 (Thermofisher, cat #A12381) and Hoechst 33342 (Thermofisher, cat #R3760), respectively, according to Obatala Sciences' standard operating procedure. The percent wound closure was evaluated using ImageJ and calculated based on the manufacturer's protocol.

### Human Subjects

Tissue specimens with procurement and ethics statements were collected from eligible donors after obtaining informed consent and were purchased from the National Disease Research Interchange (Philadelphia, PA) under protocols RGIJ1 01 001A and 002A.

FIG. 1 is a visual presentation of hydrogel #4 formulation (see table 1) for TOCNF, TOCNF (3:1) dilution, CNF and CNF (3:1) dilution after 30 minutes at 37 °C.

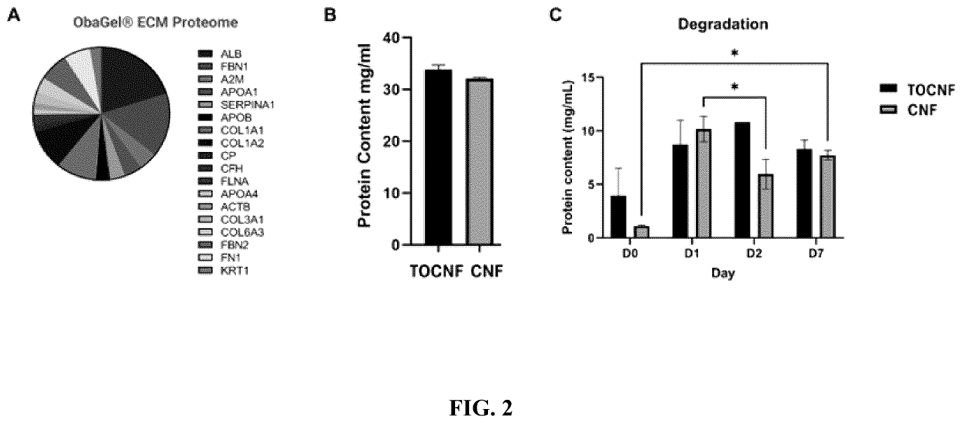

FIG. 2 shows (a) The ObaGel®-ECM proteome profile demonstrated an extracellular matrix protein dominant composition. (b) Protein content of CNF and TOCNF formulation #4 pre-crosslinked solutions determined by Pierce BCA Protein Assay as described methods section. Reported here as average ±standard deviation, n=3 technical replicates. (c) The bar graph depicts the degradation study of the hydrogel mixtures and evaluation of protein leeching. Protein content of supernatants of CNF and TOCNF formulation #4 was quantified on days 0, 1, 2, and 7 using BCA Protein Assay. Statistical significance of p<0.05 (*) between hydrogel mixtures vehicle was evaluated using a Two-way ANOVA with post-hoc Tukey's multiple comparison test. N=3 biological replicates.

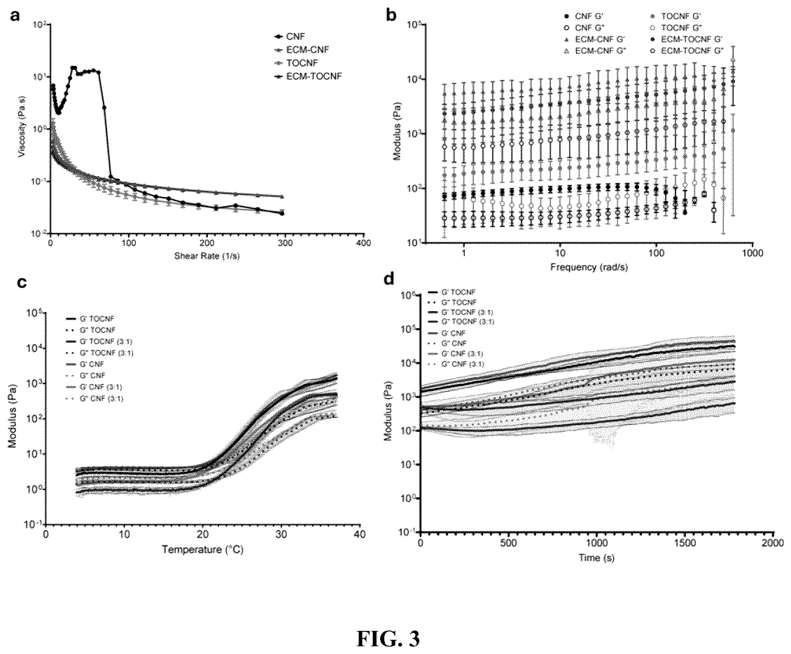

FIG. 3 shows graphical representation of the rheological data: (a) Operating viscosity of pre-crosslinked hydrogel solutions by flow ramping from 1 to 100 s⁻¹ at 4 °C, the handling temperature used was recommended to prevent premature crosslinking (n=3 replicates). CNF only solution exhibited aggregation causing a sharp increase in viscosity compared to the #4 formulation. (b) Storage (G′, solid symbols) and loss (G″, open symbols) moduli of crosslinked hydrogels by frequency sweeping from 0.1 to 100 Hz at 1% strain (n=3 replicates) of CNF and TOCNF alone compared to formulation #4 of corresponding nanofibrils. (c) For #4 formulation and its dilution, to find solution-gelation (sol-gel) transition, solutions were subjected to a temperature ramp at 1 °C/min up to 37 °C and an 1800 s post-soak at 37 °C with a 1% strain and 1 Hz frequency. Storage (G′) and loss (G″) moduli for hydrogel solutions as temperature increased during the first step and (d) Storage (G′) and loss (G″) moduli of hydrogels during the post-soak step.

TABLE 2
Storage and loss moduli and viscosity of hydrogel mixtures.

| | | Storage | Loss | |
|---|---|---|---|---|
| | Nanofibrils | Modulus | Modulus | Viscosity |
| Mixture | (w/v %) | (G′, Pa) | (G″, Pa) | (Pa · s) |
| TOCNF ctrl | 1.0% | 220.60 | 43.17 | 0.0562 |
| TOCNF #4 | 0.2% | 3309.77 | 726.36 | 0.0899 |
| TOCNF #4 (3:1) | 0.15% | 3212.47 | 602.73 | N/A |
| CNF ctrl | 1.0% | 94.23 | 29.24 | 0.0867 |
| CNF #4 | 0.20% | 6806.11 | 1976.14 | 0.0945 |
| CNF #4 (3:1) | 0.15% | 1337.29 | 284.00 | N/A |
| ObaGel ®-ECM | 0% | 64193.1 | 8131.07 | 0.0669 |

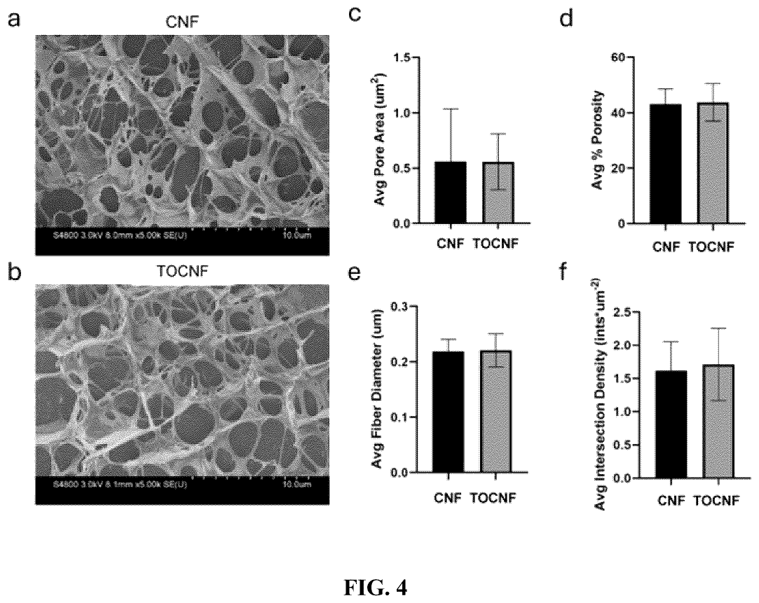

FIG. 4 shows SEM imaging and analysis revealed that both the CNF and TOCNF hydrogel mixtures exhibit similar microstructures. (a and b) SEM images of formulation #4 CNF and TOCNF hydrogel mixtures. Hydrogels were cross-linked in a 48-well plate overnight in a 37 °C, 5% CO₂ incubator, then prepared and imaged via the Hitachi S-4800 SEM equipped with Gatan Alto 2500 cryogenic SEM system the next day as described in Section 2.7. Scale bar=10 μm. (c-f) Pore area, percent porosity, fiber diameter, and intersection density were measured using Image J plugin Diameter J. Average ±SD for the set of images is reported. N=11 images analyzed for each mixture. Statistical analyses by 2-Way ANOVA with post-hoc Tukey's multiple comparison's test revealed no significant differences amongst hydrogels.

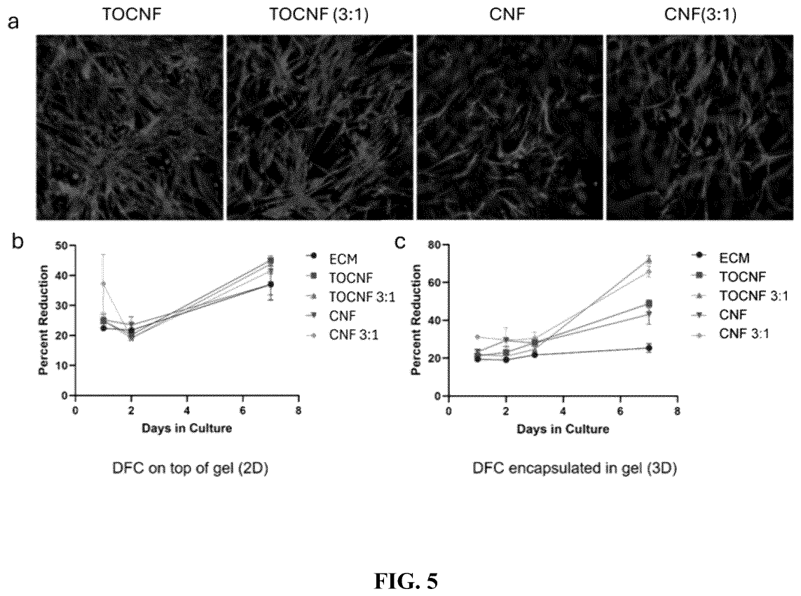

FIG. 5 shows biocompatibility, DFCs cultured on the surface or encapsulated in the CNF and TOCNF hydrogels demonstrated proliferative capacity and increased metabolic activity as represented by percent reduction of alamarBlue. (A) Representative images of DFC after 7 days of culture on hydrogel coated well plates and stained with Alexa Fluor Phalloidin 594 (red=actin) and NucBlue ReadyProbes (Hoechst 33342) (blue=nuclei). (B) Percent reduction of alamarblue was measured on days 1, 2, and 7 to assess metabolic activity of DFC cultured on top of hydrogel coated well plates. N=2 biological replicates. (C) Percent reduction of alamarblue was measured on days 1, 2, and 7 to assess metabolic activity of DFC encapsulated in hydrogels (3D). N=2 biological replicates.

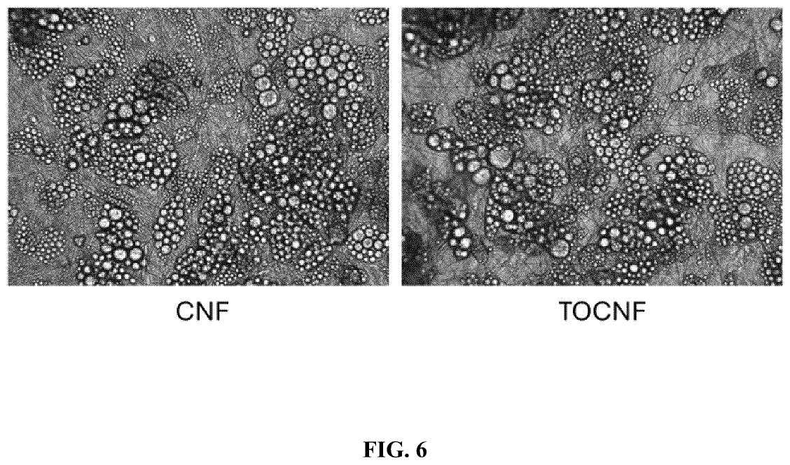

FIG. 6 shows ASCs exhibited similar lipid deposition on TOCNF and CNF hydrogels. Brightfield images of ASC cultured on PCL nanoECM fibers coated with CNF or TOCNF and maintained in adipogenic differentiation media for 31 days. 20× magnification.

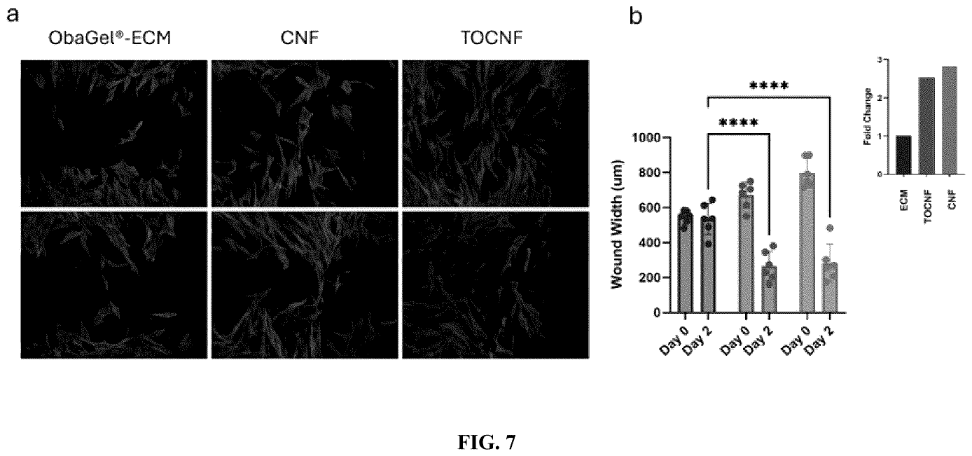

FIG. 7 shows an example of Wound Healing Assay. ASC migratory behavior is more profound on the TOCNF and CNF formulations compared to ObaGel® ECM alone. (A) Respective images of ASC cultured on ObaGel® ECM, TOCNF, and CNF coated well plate and stained with NucBlue ReadyProbes (Hoechst 33342) (blue=nuclei) and Alexa Fluor Phalloidin 594 (red=actin) at day 2 of the wound healing assay. N=2 biological replicates. 10× magnification.

Non-specific staining of fibers observed. (B) ASCs demonstrated the highest percent wound closure when cultured on TOCNF hydrogel coating. Images were analyzed using ImageJ to evaluate percent wound closure using three representative regions per image. N=2 biological replicates. Statistical analysis conducted using a 2-way ANOVA with Tukey's Multiple Comparisons Test showed significance between wound widths of ObaGel® ECM and TOCNF and ObaGel® ECM and CNF on day 2.

We claim:

1. A composition comprising: cellulose nanofibrils and at least one hydrogel wherein the hydrogel comprises at least one extracellular matrix (ECM) protein.

2. The composition of claim 1 wherein the cellulose nanofibril (CNF) is a modified TEMPO oxidized cellulose nanofibril (TOCNF).

3. The composition of claim 1 wherein the hydrogel is amended with growth factors, cytokines and at least one extracellular matrix protein selected from the group consisting of a collagen, elastin, fibronectin, laminin, glycoproteins, tenascin, fibrillin, proteoglycans and glycosaminoglycans.

4. The composition of claim 3 wherein the hydrogel comprises of a blood-derived growth factor.

5. The composition of claim 1 wherein the hydrogel comprises of a cytokine.

6. The composition of claim 3 wherein the extracellular matrix proteins include collagen and fibronectin.

7. The composition of claim 1 wherein the hydrogel comprises a proteome which supports proliferation, differentiation, and migration capacity in wound healing and tissue regeneration.

8. The composition of claim 1 wherein the hydrogel includes at least one protein selected from human serum albumin, fibrillin-1, α2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1.

9. The composition of claim 1 wherein the hydrogel comprises a proteome profile of a extracellular matrix protein composition including growth factors, cytokines and extracellular matrix proteins.

10. A method of treating a wound with an effective amount of a composition comprising: cellulose nanofibrils and at least one hydrogel wherein the hydrogel comprises an extracellular matrix protein.

11. The method of claim 10 wherein, the cellulose nanofibril (CNF) is a modified TEMPO oxidized cellulose nanofibril (TOCNF).

12. The method of claim 10 wherein, wherein the hydrogel is amended with extracellular matrix proteins.

13. The method of claim 12 wherein the hydrogel comprises of blood-derived growth factors.

14. The method of claim 12 wherein, the hydrogel comprises of cytokines.

15. The method of claim 10 wherein, the hydrogel includes at least one protein selected from human serum albumin, fibrillin-1, α2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1.

16. The method of claim 10 wherein, the hydrogel comprises a proteome which supports proliferation, differentiation, and migration capacity in wound healing and tissue regeneration.

17. The method of claim 10 wherein, the wound is a cutaneous wound.

18. The method of claim 10 wherein, the wound is a burn wound.

19. The method of claim 10 wherein, the wound is a chronic wound.

20. The method of claim 10 wherein, the wound is a wound in a diabetic patient.

21. The method of claim 10 wherein, the wound treated is decubitus ulcer.

22. The method of claim 10 wherein, the wound is a venous ulcer.

23. A method of synthesizing a wound healing composition comprising the steps of:
preparing a solution of cellulose nanofibrils in water,
adjusting the solutions to a required pH, and,
adding a extracellular matrix (ECM) hydrogel to the cellulose nanofibril solution.

24. The method of claim 23 wherein, the cellulose nanofibril (CNF) is a modified TEMPO oxidized cellulose nanofibrils (TOCNF).

25. The method of claim 23 wherein, the ECM hydrogel includes at least one protein selected from human serum albumin, collagen, fibrillin-1, α2-macroglobulin, apolipoprotein AI, α1-antitrypsin, apolipoprotein B, alpha-1 type I collagen, collagen alpha-2(I) chain, ceruloplasmin (or caeruloplasmin), carboxypeptidase H, filamin A, alpha, apolipoprotein A-IV, actin beta, type III collagen, collagen alpha-3(VI) chain, fibrillin-2, fibronectin and keratin 1.

26. The method of claim 23 wherein the hydrogel is amended with extracellular matrix proteins.

27. The method of claim 26 wherein, the hydrogel comprises of cytokines.

28. The method of claim 26 wherein, the hydrogel comprises of blood-derived growth factors, cytokines and ECM proteins.
